(19) United States
(12) Patent Application Publication
(10) Pub. No.: US 2018/0235581 A1
(43) Pub. Date: Aug. 23, 2018

(54) REMOTE AIR DETECTOR OF THE ALCOHOL CONTENT OR OTHER CHEMICAL OR NATURAL PRODUCT, HARMFUL TO THE MENTAL-PHYSICAL STATE OF A PERSON DRIVING A MEANS OF TRANSPORT

(71) Applicant: Riccarda VIANELLO, Santo Stefano Magra (SP) (IT)

(72) Inventor: Riccarda VIANELLO, Santo Stefano Magra (SP) (IT)

(21) Appl. No.: 15/772,052

(22) PCT Filed: Oct. 28, 2015
(86) PCT No.: PCT/IB2015/058313

§ 371 Date: Apr. 28, 2018

(51) Int. Cl. A61B 10/00; G01N 33/497; G01V 8/10; G01V 9/00; G01G 19/12; B60K 28/04; B60K 28/06; A61B 5/01; A61B 5/18; A61B 5/00

(52) U.S. Cl. CPC A61B 10/00; G01N 33/4972; G01V 8/10; G01V 9/005; G01G 19/12; B60K 28/04; B60K 2028/003; B60K 28/066; A61B 5/01; A61B 5/18; A61B 5/4845; A61B 5/6893; B60K 28/063

(57) ABSTRACT

A remote air detector of the alcohol content or other chemical or natural product, harmful to the mental physical state of a person driving a means of transport.

A first series of turbine suction fans for aspirating from the compartment sends the flow to an analyzer, inside the body provided with a sensor for analyzing the components of the aspirated air, a series of turbine fans for the exhalation, ejection and reintroduction of the air and any gases aspirated and analyzed. The flow rate of said first series of suction fans is higher than the flow rate of said second series of exhalation fans. Said first series of turbine suction fans is associated with unidirectional conveying fins.

Each fin is shaped substantially as an arc of circumference or alternatively ellipse, such that it occupies the outer edge of the device.

The set defined by the pair of fins directs and contains the suction cone of the driver's breath or perspiration.

The device is also provided with a distance/presence laser sensor and with a thermal sensor.

A possible weight sensor may be associated with the control and management unit of the detector device.

The present invention relates to an electro-chemical mechanical device of the “breathalyzer” type or gas detector, modified in the components and parts thereof and readapted. The device is adapted to be installed in the passenger or driving compartments of vehicles or vehicle cabs, cars, trucks, buses, trains and other means of transport.

Summarily, it comprises a breath detector with fixed installation on a mobile vehicle that allows the ethyl/alcohol vapors exhaled from the driver's breath and perspiration to be detected. The device “detects” whether the driver has a higher alcohol or drug content than permitted by law.

As will become apparent from the following description, the device object of the invention is configured to be connected to the electrical ignition system of the car (external to the controller) and is configured to actuate one or more automations and safety protocols suitable for protecting the driver, in compliance with the law regarding driving under the influence of alcohol or drugs.

A receiver sensor, such as a controller or remote control unit, placeable on the vehicle and arranged in a protected area inside the vehicle dashboard, is configured to analyze the data collected and manage the operations provided if parameters out of the levels imposed are detected.

The device can already be integrated into the vehicle upon production or it can be a separate KIT, applicable only by authorized dealers to cars or means not provided with this device.

### OBJECTS AND ADVANTAGES OF THE INVENTION

The object of the present invention is to provide an improved air detector device with a simple, reliable and cost-effective solution.

These and other objects are achieved with the features of the invention described in the independent claim 1. The dependent claims describe preferred and/or particularly advantageous aspects of the invention.

In particular, a first embodiment of the present invention provides a remote air detector device of the alcohol content or other chemical or natural product, harmful to the mental physical state, comprising a plurality of micro-fans and more precisely a first series of turbine suction fans to aspirate from the compartment and send it to an analyzer, and a series of turbine exhalation fans, to reintroduce or eject the aspirated and analyzed gases; in particular, the flow rate of the suction fans is higher than that of exhalation.

With this solution, the inlet flow is greater than the outlet flow and the analyzer has time to make an adequate analysis of the substances present in the aspirated air before it is exhausted from the detector.

A second embodiment of the present invention provides for the application of unidirectional conveying fins on the suction fans. The shape of such fins is such as to direct and contain the suction cone of the driver's breath or perspiration.

This solution with unidirectional fins allows a much more accurate detection and measurement concentrated within the driver's driving space, without detecting possible areas which would distort the data acquired, making the action of the detector inaccurate.

Another embodiment of the present invention provides, in addition to said conveying fins, a depth or distance laser sensor/meter associated with said detector. The laser sensor works so that once the detector has been placed on the vehicle, it is directed toward the driver's seat and his/her physical presence on said seat can be acquired.

With this solution, even if the person in a state of drunkenness or under the influence of drugs tries to start the engine sitting on the passenger seat, the presence laser sensor is able to communicate with the controller/control unit about the absence of the driver or the person who started the engine on the driver's seat; at that point, said control unit can prevent the starting of the vehicle and forward an SMS alert message.

Another embodiment of the present invention provides, in addition to said laser sensor/meter, also the introduction of a thermal sensor for measuring the body temperature. The thermal sensor works so that once the detector has been placed on the vehicle, it is directed toward the driver's seat and in his/her zone and his/her thermal presence on said seat can be acquired.

With this solution, even if the person in a state of drunkenness or under the influence of drugs tries to start the engine sitting on the passenger seat, the laser and thermal sensors communicate with the controller/control unit about the absence of the driver or the person who started the engine on the driver's seat; at that point, the unit controls the non-start to the vehicle controller which prevents the engine from starting, and forwards an SMS alert message.

In essence, in case of absence of driver, the car may not start, preventing the person with unacceptable values from starting the car from the other seat, away from the detector.

Another embodiment of the present invention provides for the recognition of the driver change with the vehicle engine switched on and gear disengaged: the system, continuing to detect the presence of the driver (by means of laser and thermal devices) verifies the physical condition thereof by analyzing the air by means of said analyzer sensor; in case of positive feedback, a remote message is sent to the designated security center.

In case of first start up, if the device detects a value outside the permitted limits, the device, by means of its control unit, operates the starter circuit of the vehicle, inhibiting the ability to supply current from the battery, so that starting is prevented.

Anti-tampering devices are also provided so that, if tampered with or disconnected or covered, the device equally blocks the car that cannot be started.

Moreover, upon request, a sound/light warning may be activated inside the car so as to deter the driver.

Preferably, the invention is positioned in front of the driver's driving seat, so that the distance from the latter is about 25-30 centimeters.

Especially on public means of transport, school buses, etc., it will be installed on a dedicated bracket or conformable stand.

### BRIEF DESCRIPTION OF THE FIGURES

This and other features will appear more clearly from the following description of some embodiments shown by way of a non-limiting example in the accompanying drawing tables of a remote air detector of the alcohol content or other chemical or natural product, harmful to the mental physical state of a person driving a means of transport.

FIG. 1: detail of the detector device object of the invention according to three views, from the bottom, from above and from the side.

FIG. 2: a possible application of the invention, in which the detector apparatus according to FIG. 1 is placed above the driver's seat on board the vehicle.

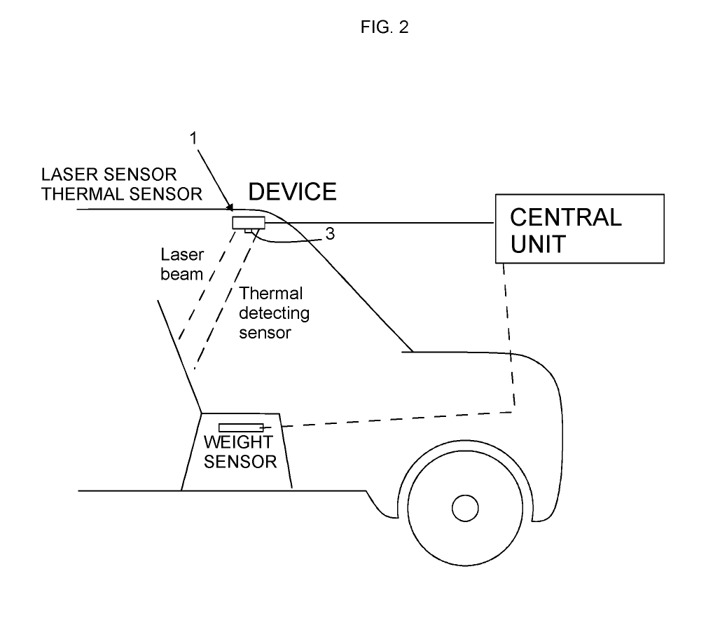

### DESCRIPTION OF THE DEVICE

The present invention will now be described in detail with reference to the accompanying drawings to enable a man skilled in the art to implement and use it. Various modifications to the embodiments described will be immediately apparent to the man skilled in the art.

With reference to the figures, reference numeral 1 indicates as a whole the apparatus for the detection of the alcohol content object of the invention, applied inside the compartment of a means of transport, in this example a motor vehicle, and precisely on the driver's side, in the upper part of the compartment, so as to:
aspirate, by means of turbine micro-fans indicated below, and
analyze, by means of an analyzer sensor provided inside or branch-connectable,
and detect:
   the micro-molecules expelled from the brow's sweat, which are also impregnated with alcoholic acidic substances if the driver has exceeded the alcohol content, or
   the alcohol vapors or other chemical or natural product, harmful to the mental physical state, from the driver, expelled from the driver's breath.

Device 1 comprises a detector body 9 having a plurality of micro-fans 2 for aspirating air inside the compartment.

More precisely, the following is provided on body 9:
a first series of turbine suction fans 2 to aspirate from the compartment (at least two in the example)
an analyzer 7, internal to the body with incoming flow analyzer sensor,
a series of turbine exhalation fans 4 (at least one), i.e. for ejecting and reintroducing the aspirated and analyzed air and any gases.

Conveniently, the flow rate of the suction fans 2 is higher than the flow rate of the exhalation fans 4. To this end, for example, two turbine suction fans and a single ejection fan are provided. Other configurations may be possible so as to have an inlet flow higher than the outlet one and give enough time to the analyzer to make an adequate analysis of the substances present in the aspirated air before it is exhausted from the detector.

The detector and analyzer device of the example has a fixed installation on the vehicle and in adequate position that allows detecting the ethyl/alcohol/drug vapors exhaled from the driver's breath or sweat.

Said detector is associated with at least one receiving sensor, such as a controller or a remote control unit; preferably, the latter is placed on the vehicle and arranged in a protected inaccessible area in the vehicle.

Said controller is connected to one or more external relays enabling horn and interior lights.

It is possible to send signals to a remote safety control center, which warns the driver of the danger of his/her physical or mental condition.

During the start up of the vehicle, in fact, the device object of the invention first performs an analysis of the air in front of the driver and checks whether the values of the aspirated ethyl vapors and sweat particles are below the predetermined threshold.

Unidirectional conveying fins on the suction fans are associated, at said turbine suction micro-fans, on the wall of the casing of device 1 or directly on the containment body of said turbine fans.

As can be seen from the figure, each fin 3 is a portion of arc of circumference or alternatively ellipse in the vicinity of the outer edge of fan 2 and which extends forward, so that the indicated pair of fins—respectively on the first and second suction fan—directs and contains the suction cone of the driver's breath or sweat.

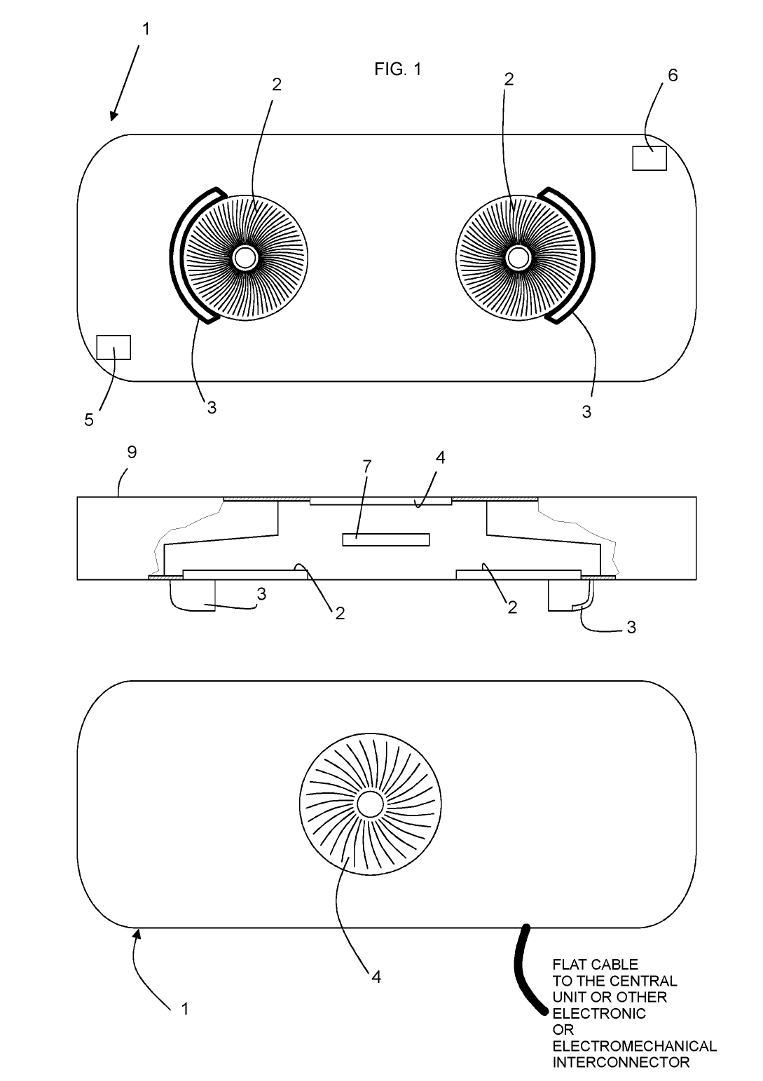

According to a median section, the fins are folded inward, as shown in FIG. 1.

In practice, acting as flow containment partitions, said unidirectional fins allow a much more accurate detection and measurement concentrated within the driver's driving space, without detecting possible areas which would distort the data acquired, making the action of the detector inaccurate.

In addition to said conveying fins 3, the presence of at least one depth or distance laser sensor/meter associated with said detector is noted.

The distance laser sensor acts so that, once the detector has been placed on the vehicle, it is directed towards the driver's seat.

With this solution, even if the person in a state of drunkenness or under the influence of drugs tries to start the engine sitting on the passenger seat, the laser sensor is able to communicate with the controller/control unit about the absence of the driver or the person who started the engine on the driver's seat; at that point, said control unit can prevent the starting of the vehicle.

In addition to said laser sensor/meter 5, the detector device is also associated with at least one thermal sensor 6 for measuring the body temperature.

Said thermal sensor works so that once the detector has been placed on the vehicle, it is directed toward the driver's seat and in his/her zone and his/her thermal presence on said seat can be acquired.

With this solution, even if the person in a state of drunkenness or under the influence of drugs tries to start the engine sitting on the passenger seat, the laser and thermal sensors communicate with the controller/control unit about the absence of the driver or the person who started the engine on the driver's seat; at that point, the unit controls the non-start to the vehicle controller which prevents the engine from starting.

On request, the management and control unit may be connected to a weight sensor placed in the driver's seat, which further verifies the presence/absence thereof.

With these safety devices, in case of absence of driver, the car may not start, preventing the person with unacceptable values from starting the car from the other seat, away from the detector.

Another embodiment of the present invention provides for the recognition of the driver change with the vehicle engine switched on and gear disengaged: the system, continuing to detect the presence of the driver (by means of laser and thermal devices) verifies the physical condition thereof by analyzing the air by means of said analyzer sensor; in case of positive feedback, a remote message is sent to the designated security center.

In case of first start up, if the device detects a value outside the limits permitted in the country where it is used, the device, by means of its control unit, operates the starter circuit of the vehicle, inhibiting the ability to supply current from the battery, so that starting is prevented.

Anti-tampering devices are also provided so that, if tampered with or disconnected or covered, the device equally blocks the car that cannot be started.

Moreover, upon request, a sound and/or light warning may be activated inside the car so as to deter the driver.

Preferably, the invention is positioned in front of the driver's seat.

1. A remote air detector device of alcohol content or other chemical or natural product, harmful to the mental physical state of a person driving a means of transport, the apparatus being associable with a receiver sensor, such as a controller or a remote control unit for processing and verifying the analyzed values, comprises:
a. at least two first turbine suction fans for aspirating from the compartment and at least one turbine exhalation fan, i.e. of ejection of said air and any gases aspirated, and wherein the flow rate of said first series of suction fans is higher than the flow rate of said second series of exhalation fans, and
b. an analyzer, internal to the body with incoming flow analyzer sensor.

2. The device according to claim 1, wherein said first turbine suction fans are associated, on the walls of the device casing or directly on the containment body of said turbine fans, a unidirectional conveying fin, respectively; each fin shaped substantially as an arc of circumference or ellipse; the fin occupies the outer edge of the respective fan so that the set defined by the pair of fins contains the suction cone of the driver's breath or perspiration.

3. The device according to claim 1, wherein the device comprises at least one depth or distance laser sensor/meter associated with said detector; said laser sensor acting so that once the detector has been placed on the vehicle, it is directed towards the driver's seat.

4. The device according to claim 1, wherein the detector device is also associated with at least one thermal sensor for detecting the body temperature; said thermal sensor acting so that, once the detector has been placed on the vehicle, it is directed towards the seat and the driver's area, and his/her thermal presence on said seat can be acquired.

5. The device according to claim 1, wherein the device provides for the recognition of the driver change with the vehicle engine switched on but gear disengaged: the system, continuing to detect the presence of the driver by laser and thermal devices verifies the physical condition thereof by analyzing the air by said analyzer sensor; in case of positive feedback, a remote message is sent to the designated security center.

6. The device according to claim 1, wherein a management and control unit also receives signals from a weight sensor placed in the driver's seat, for the verification of his/her presence/absence.
